(19) United States
(12) Patent Application Publication
(10) Pub. No.: US 2005/0079591 A1
(43) Pub. Date: Apr. 14, 2005

(54) Method and magnetic microarray system for trapping and manipulating cells

(76) Inventors: Daniel H. Reich, Baltimore, MD (US); Monica Tanase, New York, NY (US); Christopher S. Chen, Princeton, MD (US)

Correspondence Address: DOCKET ADMINISTRATOR;LOWENSTEIN SANDLER PC, 65 LIVINGSTON AVENUE, ROSELAND, NJ, 07068, US

(21) Appl. No.: 10/885,275

(22) Filed: Jul. 6, 2004

Related U.S. Application Data
(60) Provisional application No. 60/485,130, filed on Jul. 8, 2003.

(51) Int. Cl. C12N 13/00

(52) U.S. Cl. 435/173.1

(57) ABSTRACT

In accordance with the invention, a surface is provided with a plurality of microscale magnets (“micromagnets”) disposed on a surface in a pattern to form a desired distribution of magnetic field strength. Cells and magnetic nanowires are attached, immersed in fluid, and flowed over the pattern. The nanowires and their bound cells are attracted to and bound to regions of the pattern as controlled by the geometry and magnetic properties of the pattern, the strength and direction of the fluid flow, and the strength and direction of an applied magnetic field.

### CROSS REFERENCE TO RELATED APPLICATIONS

This application claims the benefit of U.S. Provisional Application Ser. No. 60/485,130 filed by Dr. Daniel Reich et al on Jul. 8, 2003 and entitled “Magnetic Microarrays For Cell Trapping and Manipulation”. The '130 application is incorporated herein by reference.

### GOVERNMENT INTEREST

This invention was made with government support under DARPA/AFOSR Grant No. F49620-02-1-0307 and by NSF Grant No. DMR-0080031. The government has certain rights to this invention. The work was also supported by the David and Lucile Packard Foundation.

### FIELD OF THE INVENTION

This invention relates to a method and system for trapping and manipulating biological cells.

### BACKGROUND OF THE INVENTION

Methods of trapping and manipulating biological cells are highly important in a wide variety of applications including rapid diagnostic procedures, cell separation, isolation of single cells, control of cell-cell interactions, tissue engineering and biosensing. For example, many rapid diagnostic techniques require rapid controlled spreading of cells for optical scanning. Analyses of rare DNA require isolation of single cells for investigation, and trapping clusters of a determined number of cells is important for controlling and studying cell-cell interactions and biological functions in the presence of neighboring cells.

One approach to obtaining a desired cell pattern is to provide a substrate chemically patterned with regions of cell-adhesive ligands in alternation with non-adhesive regions. A cell suspension is placed in contact with the substrate and cells adhere to the ligand regions. Unfortunately the adhesion process is slow, also the process is irreversible, which is inconvenient for some applications.

Another approach is dielectrophoretic trapping. Strong high-frequency AC electric field gradients from shaped electrodes move cells by coupling to dipole moments induced in the cells. The technique, however, requires a low conductivity culture medium and presents the complexity of working with strong, high frequency fields.

Yet another approach useful in cell separation is the use of micron scale magnetic beads. Magnetic beads with ligands that will selectively bind to one cell type are added to a suspension of mixed cells. Cells of the chosen type will attach to the beads, and the beads with their attached cells can be magneticially separated from the suspension. This technique is useful for separation but is limited in speed and manipulative capability. Accordingly there is a need for an improved method and system for trapping and manipulating cells.

### SUMMARY OF THE INVENTION

In accordance with the invention, a surface is provided with a plurality of microscale magnets (“micromagnets”) disposed on a surface in a pattern to form a desired distribution of magnetic field strength. Cells and magnetic nanowires are attached, immersed in fluid, and flowed over the pattern. The nanowires and their bound cells are attracted to and bound to regions of the pattern as controlled by the geometry and magnetic properties of the pattern, the strength and direction of the fluid flow, and the strength and direction of an applied magnetic field.

### BRIEF DESCRIPTION OF THE DRAWINGS

The advantages, nature and various additional features of the invention will appear more fully upon consideration of the illustrative embodiments now to be described in detail in connection with the accompanying drawings. In the drawings:

FIG. 1 is schematic block diagram of the steps involved in trapping and manipulating biological cells in accordance with the invention;

FIG. 2 is a schematic diagram illustrating magnetic trapping of cells attached to nanowire carriers;

FIGS. 3 and 4 show exemplary apparatus for trapping and manipulating cells;

FIGS. 5a and 5e are a scanning electron micrographs (SEM's) illustrating nanowires and nanowire bound cells.

FIGS. 6a through 6c illustrate magnetic cell chaining in accordance with the invention;

FIGS. 7a through 7c illustrate magnetic trapping of single cells in accordance with the invention;

FIGS. 8a through 8o are overview images of cell trapping on magnetic arrays and associated magnetic field energies;

FIGS. 9a through 9f illustrate directed cell chain formation due to nanowire-nanowire interactions;

FIG. 10 shows magnetic trapping under diagonal fluid flow; and

FIGS. 11a through 11d illustrate magnetic trapping effected by reversing an external magnetic field.

It is to be understood that these drawings are for illustrating the concepts of the invention and, except for the graphs, are not to scale.

### DETAILED DESCRIPTION

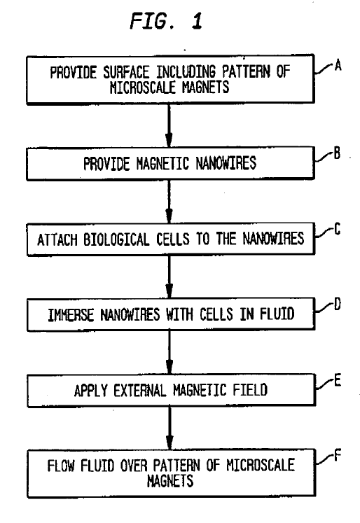

Referring to the drawings, FIG. 1 is a schematic block diagram of the steps involved in trapping and manipulating biological cells in accordance with the invention. A first step, shown in Block A, is to provide a surface including a pattern of microscale magnets arranged to form a desired distribution of magnetic field strength over the surface. By the term “microscale magnet” is meant a magnet having maximum dimensions of less than 1 mm in each of the three dimensions. The preferred microscale magnets (“micromagnets”) are planar structures in the shape of ellipses or ovals formed on the surface. Typical micromagnet dimensions are length of 80 micrometers, width of 8 micrometers and thickness of 0.4 micrometers. Other shapes and dimensions may be used and can be manufactured by photolithographic techniques well known in the art.

When magnetized, the micromagnets behave as tiny permanent magnets. They each produce a magnetic field which has the general field configuration produced by a magnetic dipole. When the micromagnets are disposed in close proximity, their magnetic fields overlap and add together. Thus by appropriate distribution of the magnets one can achieve a desired distribution of magnetic field strength over the support surface and, in particular, a plurality of regions of relatively high field strength. An advantageous pattern comprises an array of neighboring spaced apart micromagnets. When the north magnetic pole of a magnet is close to the south pole of a neighboring magnet, there are strong magnetic fields in the region between the two neighboring magnets.

The next step, shown in Block B, is to provide a plurality of magnetic nanowires to act as carriers of the biological cells. By the term “nanowire” is meant a structure having maximum dimensions of less than about one micrometer in two of the three dimensions (the transverse dimensions) and a maximum third dimension (the longitudinal dimension) that is larger, preferably by a factor of 10 or more. Advantageously the nanowires have transverse dimensions (typically diameters) in the range 20 to 500 nanometers and longitudinal dimensions of 500 nm to 50 micrometers. The transverse cross section of the nanowire can be round, tubular, rectangular or any desired shape. The nanowire carriers can be formed, for example, by electrodeposition of magnetic material such as nickel, into a nanoporous template (e.g. aluminum oxide) and removal of the template material, as by etching it away.

The third step (Block C) is to attach the biological cells and the magnetic nanowires. Advantageously the nanowires and biological cells are attached by inclusion of the nanowires in the cells. A protocol for attachment is described herein below. An alternative approach is to bind to the nanowires a material such as transferrin that stimulates cell intake of the nanowires. Yet further in the alternative, the nanowires can be externally attached to the cells, as by chemical bonding to a cell receptor.

The next step shown in Block D is to immerse the nanowire carriers with attached cells in fluid.

An external magnetic field is applied to orient the suspended nanowire carriers (Block E), and the fluid is flowed over the pattern of micromagnets (Block F). With appropriate patterns of micromagnets and flow rates, carriers with cells are trapped in regions of high, compatibly oriented magnetic field strength.

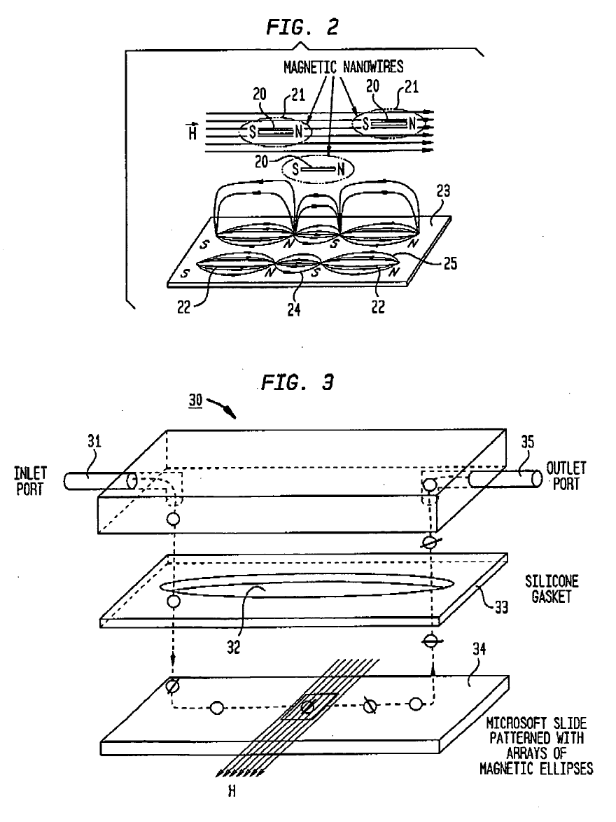

FIG. 2 is a schematic diagram illustrating the magnetic trapping. Magnetic nanowires 20 with cells 21 are suspended in a fluid (not shown) and travel with their magnetic poles (N,S) lined parallel to the externally applied field H.

The micromagnets 22 on support surface 23 generate regions 24 of high magnetic field polarity compatible with the magnetic orientation of the aligned carriers 20 and regions 25 of polarity incompatible with the carrier orientation. The nanowires 20 are attracted to the compatible regions 24 and repelled from the incompatible regions 25.

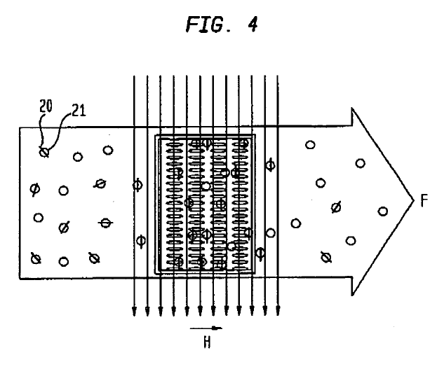

The mechanisms for manipulating the carried cells can be further understood by consideration of FIGS. 3 and 4 which illustrate exemplary apparatus 30 for trapping and manipulation. FIG. 3 is an exploded schematic view of the apparatus 30 comprising a fluid inlet port 31, a fluid flow channel 32 defined, for example, in an elastomeric gasket 33, a surface 34 supporting an array or pattern of microscale magnets (not shown) and a fluid outlet port 35. An external magnet (not shown) supplies an external magnetic field H in the flow region near the surface 34. The channel is typically about 100 micrometers deep. As better shown in FIG. 4, the magnetic field H is advantageously substantially transverse to the direction of fluid flow F.

The external field plays an important role in trapping and manipulation. If, for example, the external field is realigned to orient the suspended nanowires with their magnetic poles in opposition to the poles of the micromagnets, then instead of being attracted to the ends of the magnets, the nanowires and any cells bound to them will be attracted and bound on top of the micromagnets just as bar magnets brought together with their poles oriented in opposition. Thus after trapping one type of cell between micro magnets, the external field can be reversed and a second type of cell can be trapped on top of the magnets to create interpenetrating bands of different cell types.

The fluid flow provides a controlled method of introducing cells onto the array. At high flow rates, the forces on the nanowire carriers produced by the moving fluid will exceed the magnetic forces between the carriers and the array, and few if any cells will be trapped by the array. At lower flow rates, more detailed control of trapping can be achieved. The relative occupation of trapping sites by multiple cells, as opposed to single cells, varies with the fluid flow rate. The direction of flow provides another parameter for control. It can cause cells to be preferentially trapped on the upstream ends of the micromagnets.

The invention may now be more clearly understood by consideration of the following specific examples.

### EXAMPLE 1

### Fabrication of Nanowire Carriers and Attachment of Cells

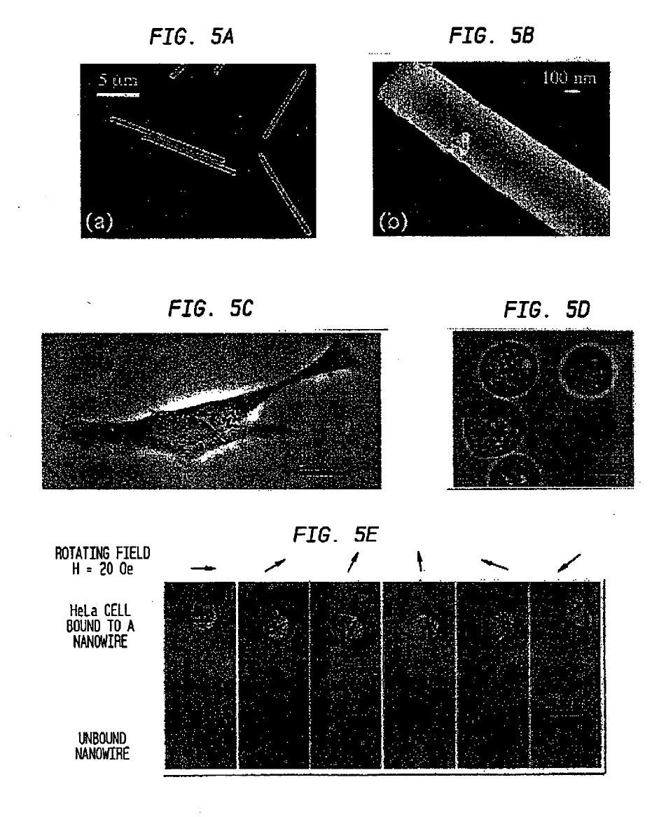

Sample fabrication. Nickel nanowires were fabricated by electrochemical deposition in the cylindrical nanoporous of 50 μm-thick alumina filtration membranes (Anodisc, Whatman, Inc.). The wires' radius r_{w}=175±20 nm was determined by the pore size, and their length was controlled by monitoring the deposition current. After deposition, the alumina was dissolved in 50° C. KOH, releasing the nanowires from the membranes. Once in suspension, the wires were collected with a magnet, washed with deionized water until the pH was neutral, then sterilized in 70% ethanol and suspended in 1× phosphate buffered saline solution (PBS). In the course of this process the wires were exposed to large magnetic fields in excess of 0.3 T. Due to their large magnetic shape anisotropy, they subsequently remained highly magnetized with a remnant magnetization M_{W}≈330 kA/m which is 70% of their saturation magnetization. A scanning electron micrograph of several wires is shown in FIG. 5a, and a close-up of a portion of a single wire is shown in FIG. 5b.

For the magnetic cell trapping studies, arrays of permalloy (Py, Ni₇₁Fe₂₉) micromagnets were fabricated on glass microscope slides or cover slips. Py films 400 nm thick were deposited by magnetron sputtering, and the micromagnets were produced by standard contact photolithography and chemical etching in 10% wt. nitric acid. The individual micromagnets were elliptical in shape, with major axis α=80 μm, and minor axis β=8 μm. This shape gives well-localized trapping sites at the ends of the ellipses. It also minimizes the formation of multi-domain configurations within individual micromagnets that could broaden the distribution of the micromagnets' magnetic moments. Rectangular arrays containing up to 4000 ellipses were fabricated in 5×5 mm² fields. The lattice constants (center-to-center spacings between elements) of the arrays were in the range 110 μm≦a≦40 μm in the direction parallel to the ellipses' major axes, and 17 μm≦b≦100 μm along their minor axes. The magnetization curves of the micromagnet arrays were measured in a vibrating sample magnetometer. In the 10 mT fields used in the trapping experiments, the ellipses have magnetization M_{E}=650 kA/m, and magnetic moment μ_{E}=1.3×10⁻¹⁰ A·m²/ellipse.

Cell culture. NIH-3T3 mouse fibroblasts cells (ATCC, USA) were cultured at 37° C., 5% CO₂ in Dulbecco's Modified Eagle Medium (DMEM) (Gibco Life Sciences) supplemented with 1% penicillin/streptomycin and 5% calf serum. HeLa cells were grown under similar conditions in DMEM with 10% fetal bovine serum, but without antibiotics. The nanowires were introduced into the culture dishes when the cells were at 40% confluence at concentrations of at most 1 wire per 3 cells to reduce the probability of multiple wires binding to the same cell. The extracellular matrix proteins present in the serum-enriched media adsorb to the hydrophilic native oxide layer on the surface of the wires, and promote non-specific binding of the nanowires to the cells. The wires and cells were incubated together for 24 hrs, at which point the number of unbound nanowires was observed to become minimal. FIG. 5c shows a nickel nanowire bound to a 3T3 cell in culture. Previous studies have shown that Ni nanowires do not have toxic effects on 3T3 cells over periods longer than the duration of the current experiments.

### EXAMPLE 2

### Magnetic Manipulation of Cells

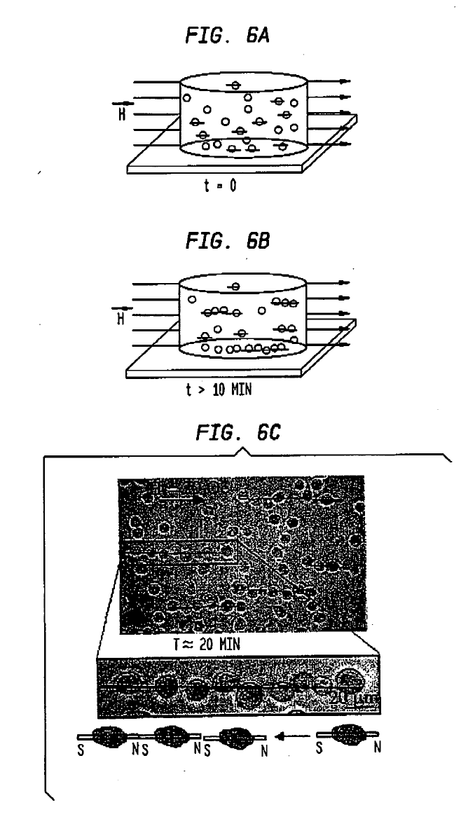

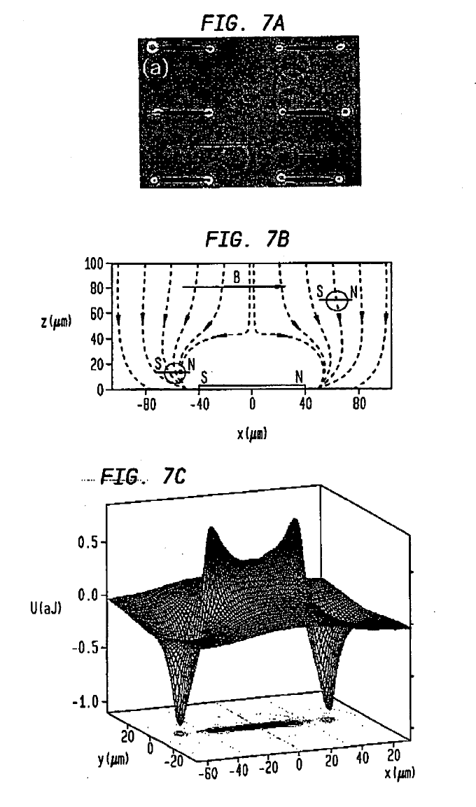

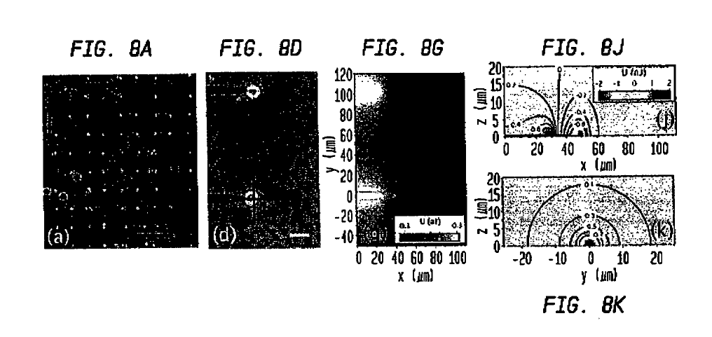

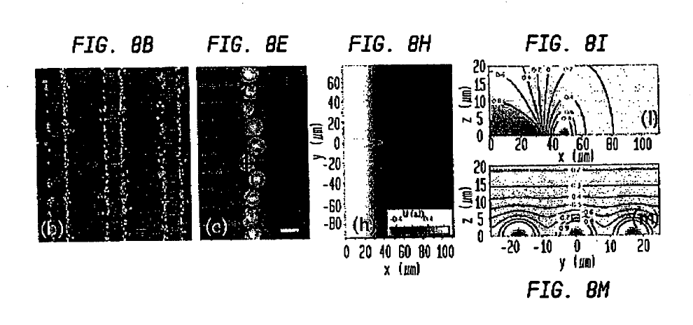

For the magnetic manipulation experiments the cells were detached from the culture dishes using 0.25% trypsin and 1 mM ethylenediaminetetraacetic acid in PBS, and re-suspended in fresh culture medium. The wire-cell binding is quite robust, and is resilient to the exposure to trypsin [Hultgren04]. Cells without wires were removed by a single-pass magnetic separation [Hultgren03] to increase the fraction of cells bound to a wire to 75%. A suspended 3T3 cell with a bound wire is shown in FIG. 5d. For the cell chaining experiments, 1 ml aliquots of cell suspensions with number densities in the range 1×10⁵-2.5×10⁵ cells/ml were placed in 1.8 cm² rectangular culture dishes. A uniform external field B=2 mT was applied to align the wires, as shown schematically in FIG. 6a, and chain formation was monitored as the cells settled to the bottom of the dish (FIG. 6b).

The cell trapping experiments were carried out either by sedimentation onto the micromagnet arrays under similar conditions as for the chaining experiments, or using a fluidics apparatus based on previously reported designs. For this flow-assisted trapping, a microscope slide patterned with micromagnet arrays formed the bottom of a parallel-plate flow chamber with width w=6 mm, height t=100 μm, and length L_{C}=2.5 cm. The arrays were oriented with the long axes of the micromagnets perpendicular to the flow direction. The chamber's inlet and outlet ports were connected through multi-port valves to 10 ml syringes which served as fluid reservoirs. The chamber was sterilized with 70% ethanol, and rinsed with DI water and culture medium before introduction of cells. Cell suspensions with number densities of 2.5×10⁵ cells/ml were introduced into the chamber at constant flow rates QF in the range 0.5≦Q_{F}≦7.5 μL/s using an injection/withdrawal syringe pump (Model M362, Thermo Orion). A uniform external field B=10 mT was applied parallel to the micromagnets' long axis. This field both magnetized the micromagnets, and aligned the wires with their moments parallel to that of the micromagnets.

Trapping and chain formation were recorded in both phase contrast and bright field with the 10× and 40× objectives of a Nikon Eclipse TS100 inverted microscope equipped with a digital camera (Nikon Coolpix 995E) and video acquisition system. Higher-resolution phase contrast images of single cells with wires were obtained with the 20× objective of a Nikon TE2000 microscope, and reflected light images of cells trapped on top of micromagnets were taken with the 10× objective of a Nikon Labphot upright microscope.

Due to their large magnetic moment, nanowires maintain their responsiveness to small magnetic fields, even when bound to cells. The torques produced on the wires by the 2-10 mT external uniform fields employed here are more than enough to line up the wires bound to cells with the field direction. This is illustrated in FIG. 5e, where a HeLa cell is rotated into a number of different orientations by varying the direction of the applied field.

### EXAMPLE 3

### Chain Formation

FIG. 6 shows a chain-formation experiment. Here, an external field aligns the wires' moments parallel to the field and to each other as sketched in FIG. 6a and 6b. The cells descend through the culture medium with a sedimentation velocity of approximately 6-10 mm/h, and the nanowires experience mutually attractive dipole-dipole forces due to the interactions of their magnetic moments. The alignment of the wires makes it unfavorable for wires to approach each other side by side, and favors the formation of head-to-tail chains, where the North pole of one wire abuts the South pole of the next. Chains of cells become detectable approximately 10 min into the experiment, and as shown in FIG. 6c, these formations can encompass many cells, and extend over hundreds of micrometers. Cells without wires settle at random. We observe two mechanisms of chain formation: aggregation in suspension, which leads to short chains, and the addition of descending individual cells or short chains to pre-existing chains on the chamber bottom. The chaining process ceases once all cells have settled because the interwire forces are not sufficiently strong to cause the 3T3 cells to move along the substrate.

### EXAMPLE 4

### Magnetic Trapping

When cells with wires are brought in proximity to patterned micromagnet arrays either by sedimentation or by fluid flow, they are attracted to the ends of the ellipsoidal micromagnets where the local field is most intense. This is shown in FIG. 7a, where 3T3 cells have been trapped at the ends of six ellipses. The sedimentation trajectories calculated for a cell with a wire are displayed in FIG. 7b. Models for calculating the magnetic forces and the sedimentary trajectories are set forth in Appendix A hereto.

### EXAMPLE 5

### Flow-Assisted Trapping

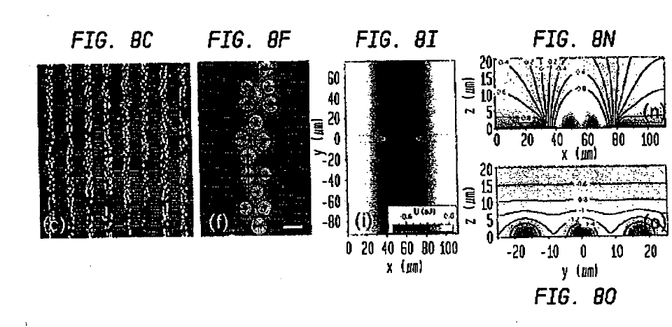

While trapping of cells can be achieved by sedimentation, it is much faster and more efficient to use fluid flow to bring the cells onto the arrays. Some of the cell patterns achievable with flow-assisted trapping are shown in FIG. 8. FIG. 8a shows a sparse array with center-to-center spacings a=206 μm and b=102 μm. The predominant mode of trapping is the capture of single cells, as is shown in more detail in FIG. 8d. The array in FIG. 8b contains well-separated columns of closely spaced ellipses, with a=338 μm, and b=17 μm. A close-up of this array is shown in FIG. 8e. Here, the trapping process induces formation of lines of cells along the edges of the columns. The spaces between the columns are swept clear of cells by the fluid flow. FIG. 8c and the corresponding high-magnification image in FIG. 8f show that when columns such as those in FIG. 8b are brought into close proximity, with horizontal spacing a=112 μm, sharply defined stripes of cells are formed. Formation of these patterns depends critically on the repulsion of cells with wires from the regions over the micromagnets. Cells without wires are removed from all types of arrays by the fluid flow, and as can be seen in FIGS. 8d-f, all of the trapped cells have wires.

As the cells approach the array, the large-scale features of the pattern they will form is determined by the field profile generated by the array well above the substrate. The grayscale magnetic energy maps in FIGS. 8g-i correspond to the regions of the arrays shown in FIGS. 8d-f, and were calculated for a wire of length L=20 μm at height z=8 μm above the substrate, a distance equal to the average suspended cell radius. At this height, in the sparse arrays, the attractive wells with U<0 from the individual ellipses, which appear as dark spots, are spatially separated and have sizes comparable to a single cell, as shown in FIG. 8g. In the denser arrays, however, the wells overlap and reinforce each other, forming regions resembling trenches running parallel to the columns of ellipses that attract the wires more strongly than do the individual ellipses. For widely separated columns the trenches are narrow as in FIG. 8h, leading to the capture of lines of cells as seen in FIG. 8e, while for close-packed columns the trenches are wider, as in FIG. 8i, and stripes of cells form such as those in FIG. 8f. Note also that the regions with U>0 over the ellipses (shown in white) coalesce over the columns in FIGS. 8h and 8i, leading to large areas of the arrays from which the cells are repelled and pushed toward the regions where trapping is favored. Indeed the dense arrays are more efficient at trapping cells, as they always subject a cell with a wire to either attractive or repulsive interactions, whereas by contrast, when flowing over more sparse arrays, the wire scans large neutral areas.

FIGS. 8j-8o show color-coded magnetic energy maps for 20 μm wires in the x-z and y-z planes above the horizontal and vertical lines in FIGS. 8d-8f. The x-z maps over the centerline of an ellipse show that there is a strongly localized binding site for a wire with its end just touching the end of the ellipse. The ellipses appear as black bars at the bottom of these figures. Note that the color scale has been truncated, and the calculated depths of the wells are U_{Min}=−6.4 aJ, −6.6 aJ, and −7.1 aJ for FIGS. 8d-8f, respectively. The y-z maps cut through these binding sites, and show that these sites are well localized at the tip of each ellipse. This shows that the final position of the cells within the attractive regions on the array is predominantly determined by the interaction of the wire with a single ellipse, and explains the registry of the cells within the trenches in FIGS. 8e and 8f. The repulsive regions (blue) extend to much higher altitude in the close-packed arrays, which contributes to their greater efficiency at trapping.

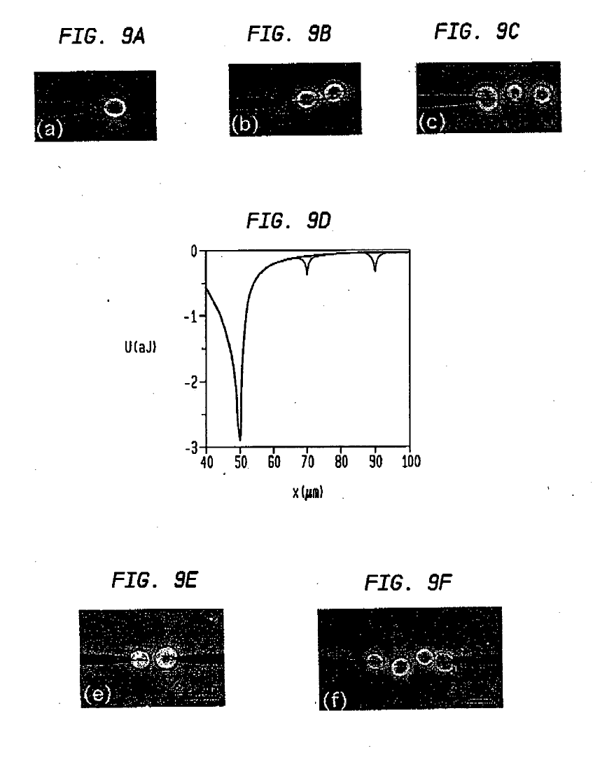

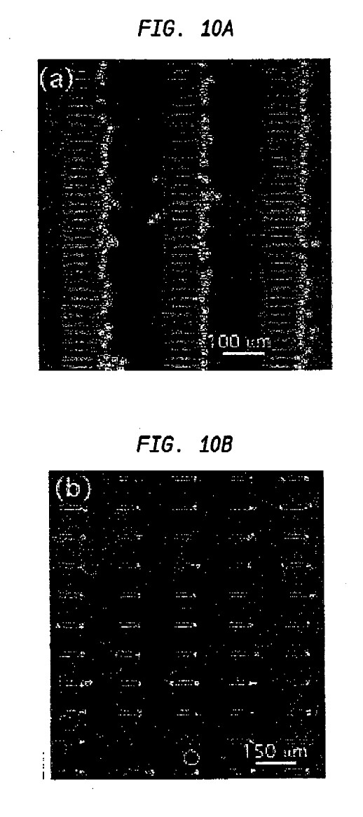

Once a cell is trapped at a micromagnet, subsequent cells are prevented from trapping at that end of the ellipse by volume exclusion. This occupation of the trapping sites contributes to the quasi-regular positioning of the cells in the lines and stripes shown in FIGS. 8d and 8g respectively. At the same time, the presence of a trapped wire such as that in FIG. 9a modifies the magnetic energy surface seen by subsequent cells with wires, and if space allows, these cells will form chains with their wires lined up with the wire of the first trapped cell, as shown in FIGS. 9b and 9c. To model this chain formation at the ends of the micromagnets, the contribution of trapped wires to the magnetic energy of an incoming wire must be included. The black line in FIG. 9d shows the variation of the wire-ellipse interaction energy U₁ along the ellipse's centerline with distance from the end of the ellipse at z=0.4 μm. A 20 μm wire trapped by this ellipse with its center at x=50 μm produces a secondary energy minimum, as shown by the red curve in FIG. 9d, which was calculated for the purposes of illustration for a second 20 μm wire at a height Δz=2r_{W} above the first wire. Once trapped, the second wire produces a third energy well, shown in blue, that can trap a third wire-cell pair, leading to the situation shown in FIG. 9c.

The length of these chains can be controlled by the horizontal spacing between the micromagnets in the array. FIG. 9e shows a gap sized for trapping pairs of cells, and FIG. 9f shows a chain of length four. Thus the micromagnets can serve as localized initiation sites for cell chain formation, with the spacing between the micromagnets controlling the number of cells in the chains.

### EXAMPLE 6

### Effects of Fluid Flow

The speed and direction of the fluid flow in the chamber further controls of the geometry of the trapped cell patterns. The fluid force f_{F} on the cells affects both the trapping efficiency and the occurrence of chaining. The images shown in FIGS. 7-9 were obtained at flow rates 0.5 μl/s≦Q_{F}≦1.7 μl/s. These low flow rates favor high trapping site occupancy (>80%), as well as formation of cell chains. Above Q_{F}˜1.7 μl/s, first chaining and then trapping were incrementally suppressed, and at the highest flow rate measured, Q_{F}˜7.5 μl/s, chain formation was virtually absent, and only 10% of the sites were occupied by cells. Since the trapping must overcome the fluid force on the cells, increasing f_{F} reduces the range of influence the trapping sites, and raises the threshold for the magnetic force required to capture cells. From the hydrodynamic force on a sphere near a surface under laminar flow, we obtain f_{F}≈1.5 nN at Q_{F}=7.5 μl/s. This is equal to the calculated peak wire-wire force F_{ww}, and explains the suppression of chain formation at higher flow rates. The peak wire-trap force F_{Tr}≈22 nN considerably exceeds f_{F}, consistent with our observation that once a cell was trapped by a micromagnet, even our highest constant flow rates were not sufficient to remove it. However, by pulsing the inlet syringe briefly it was possible to dislodge the cells from the traps, and thus the magnetic trapping process can indeed be made reversible.

The cell patterning can also be controlled by the direction of the flow relative to the arrays. When the flow was angled more than 5° from perpendicular to the long axes of the ellipses, we obtained strong preferential trapping on the upstream ends of the ellipses, as shown in FIG. 10. This occurs because the incoming cells are blocked from reaching the downstream trapping site of each by the repulsive region above the ellipse's center. The diagonal flow also ensures that most cells will reach the proximity of at least one trapping site while traversing the array, and the trapping efficiency per upstream site is therefore increased over perpendicular flow. Compare FIG. 10(a) where 94% of the upstream sites are occupied and only 3% of the downstream sites, to FIG. 8(b) where under perpendicular flow, the overall occupancy rate is 78%. This selectivity is also observed in sparser arrays, as shown in FIG. 10(b) where 83% of the upstream sites were occupied, and only 25% of the downstream sites. Another important result achieved here is the large number of single cells obtained on the sparse array: approximately 800 across a 5×5 mm² area in less than 10 minutes. This potentially useful in applications that require the interrogation of spatially separated individual cells.

### EXAMPLE 7

### Effects of Field Reversal

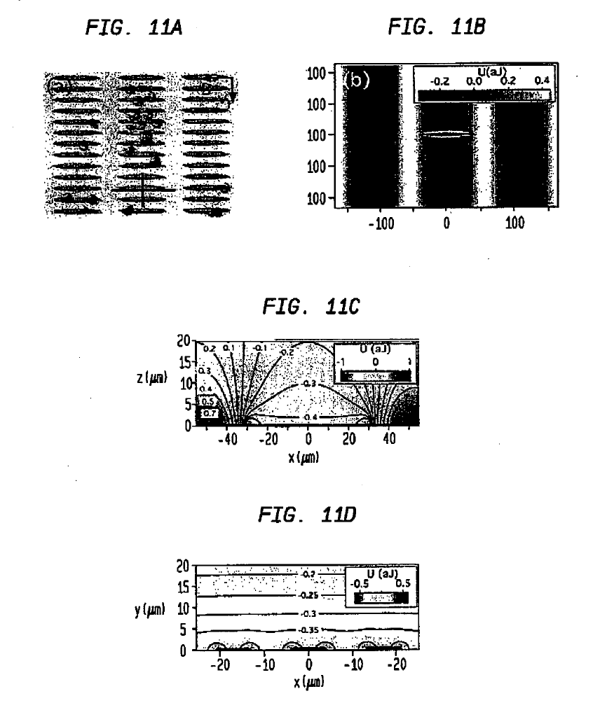

Trapping experiments were also performed with the direction of the applied field reversed. Care was taken to not exceed the coercive field μ₀H_{C}=2 mT at which the magnetization of these micromagnets reverses. Consequently, the wires' moments were antiparallel to those of the micromagnets, and wire-micromagnet interaction changed sign. It then became favorable for cells with wires to land on top of the micromagnets, rather than at the ends. This is shown in FIG. 11a, with B=−0.5 mT. This inversion is also apparent from the calculated wire-ellipse interactions. The stripes of attraction at z=8 μm are now located over the columns of ellipses, as shown in FIG. 11b, and there is a broad attractive region at lower altitude over the whole column, as shown in the x-z map along the centerline of one ellipse in FIG. 11c, and the y-z map over the center of a column shown in FIG. 11d. The trapping is somewhat less effective in this case, as the weaker modulation compared to the parallel trapping shown in FIG. 8 leads to less well-defined trapping sites with weaker binding. This weakening of the trap was further exacerbated in our experiments by the low remnant magnetization of our Py ellipses which results in M_{E}≈300 kA/m at B=−0.5 mT. The resulting weaker magnetic forces placed an upper limit on the flow rate of Q_{F}<0.15 μL/s for effective trapping. Thus, while these experiments demonstrate the potential to trap cells on top of the ellipses, this approach can be improved by constructing the micromagnets from magnetically harder materials.

### Conclusions

We have shown that magnetic nanowires used in conjunction with micropatterned magnetic arrays provide a flexible tool for manipulation and positioning of cells. Due to their large remenant magnetic moment, the nickel nanowires used are very responsive to small fields, even when bound to a cell. The nanowires were shown to mediate self-assembly of cell chains due to wire-wire interactions. Trapping and positioning of cells bound to wires was achieved using arrays of patterned micromagnets. This process can be precisely modeled based on dipolar interactions between the wires and the micromagnets, and therefore a wide variety of potentially useful geometries can be readily engineered. This magnetic cell patterning was shown to be controllable through a combination of external magnetic fields and fluid flow. In particular, the ability to invert the sign of the wire-micromagnet interaction at any time by a simple reversal of the field direction has the potential to enable controlled assembly and spatial positioning of multiple cell types or other heterogenous configurations without the use of selective functionalization or other chemical modification of the substrate. Ultimately, the ability to use magnetic nanowires to bring large numbers of cells to precise locations in a custom-engineered environment should enable their use in a variety of research, diagnostic and biosensing applications.

It is to be understood that the above-described embodiments are illustrative of only a few of the many possible specific embodiments that can represent applications of the invention. Numerous and varied other arrangements can be made by those skilled in the art without departing from the spirit and scope of the invention.

### Appendix A: Physical Models Relevant to Magnetic Trapping

Magnetic trapping. When cells with wires are brought in proximity to patterned micromagnet arrays either by sedimentation or by fluid flow, they are attracted to the ends of the ellipsoidal micromagnets where the local field is most intense. This is shown in FIG. 7a where 3T3 cells have been trapped at the ends of six ellipses.

We calculated the magnetic forces driving the cell trapping from the magnetostatic interactions between the wire and the micromagnet array. As the 10 mT external field oriented the wires nearly parallel to the ellipses' major axis, the force on a wire due to a single ellipse was

$${F}_{1} = - {\int }_{wire}^{} ( ∇ {B}_{E}^{x} ) ⅆ {μ}_{w} .$$

Here B_{E}^{x} is the component of the ellipse's magnetic field parallel to the wire (we use a coordinate system with \hat{x} parallel to the ellipses' major axis and \hat{z} vertical) and dμ_{w}=M_{w}dV is the dipole moment of a volume element dV of the wire. Sufficient accuracy was obtained by treating the wires as one-dimensional objects with moment per unit length πr_{w}²M_{w}. B_{E}^{x} and ∇B_{E}^{x} were calculated from the bound surface current density on the ellipse using the Biot-Savart law F₁ was computed numerically on a 0.5 μm mesh, and the total magnetic force F_{M} on a wire at position r above an array was obtained to better than 0.1% accuracy via interpolation of the computed values of F₁ as

$${F}_{M} ( r ) = {\sum }_{n , m} {F}_{1} ( r - {R}_{n , m} )$$

where R_{n,m}=na\hat{x}+mbŷ gives the positions of the micromagnets in the arrays.

FIG. 7b displays sedimentation trajectories calculated for a cell with a wire settling over the centerline of an isolated ellipsoidal micromagnet. As all motion in these experiments occurs at low Reynolds number, the velocity field that determines these trajectories is v=F_{T}/ζ, where ζ is the appropriate drag coefficient, and F_{T} includes both magnetic and gravitational forces. Those shown were calculated for a 16 μm diameter spherical cell with average density 1.08 gm/cc bound to a 20 μm wire. The concentration of the trajectories illustrates the attractive action of the trap. Note the excluded region extending approximately 40 μm above the micromagnet. Such calculations are in good agreement with the observed motion of the cells.

The magnetic energy U(r) of a nanowire over an array is useful in visualizing how the cells with wires are trapped on the arrays. In a similar manner as for the force, this was calculated from

$$U ( r ) = {\sum }_{n , m} {U}_{1} ( r - {R}_{n , m} ) . \\ where$$

$${U}_{1} = - {\int }_{wire}^{} {B}_{E}^{x} ⅆ {μ}_{w} ,$$

is the energy of a wire interacting with a single ellipse. A map of U₁ with a nanowire at height z=3 μm above the substrate is shown in FIG. 7c. Note the repulsive region with U₁>0 located over the ellipse, and the deep, attractive wells with U₁<0 at each end of the ellipse. These calculations demonstrate an important feature of the trapping, namely that with μ_{W}∥μ_{E} the cells with wires are strongly repelled from the centers of the micromagnets and never land there.

1. A method of manipulating and trapping biological cells comprising the steps of:
providing a surface including thereon a plurality of magnets arranged in a pattern to form a desired distribution of magnetic field strength over the surface including one or more regions of relatively high field strength;
providing a plurality of magnetic nanowires to act as carriers of the biological cells;
attaching together the magnetic nanowires and the biological cells and;
immersing the nanowires and attached cells in fluid and applying the fluid over the pattern of magnets on the surface, thereby attracting nanowires and attached cells to compatible regions of high field strength.

2. The method of claim 1 further comprising the step of applying an additional magnetic field to orient magnetic nanowires immersed in the fluid.

3. The method of claim 1 further comprising the step of flowing the fluid across the pattern of magnets on the surface.

4. The method of claim 2 further comprising the step of varying the strength or direction of the additional magnetic field.

5. The method of claim 3 further comprising the step of varying the rate or direction of the fluid flow.

6. The method of claim 1 further comprising the steps of:
applying an additional magnetic field to orient the magnetic nanowires immersed in the fluid; and
flowing the fluid over the pattern of magnets on the surface.

7. The method of claim 6 further comprising the step of varying the direction of fluid flow in relation to the direction of the additional magnetic field.

8. The method of claim 6 further comprising varying the rate or direction of fluid flow.

9. The method of claim 6 further comprising varying the the strength or direction of the additional magnetic field.

10. The method of claim 1 wherein the magnets comprise microscale magnets having maximum dimensions of less than 1 millimeter in each of the three dimensions.

11. The method of claim 1 wherein the magnetic nanowires have maximum transverse dimensions of less than one micron and maximum longitudinal dimensions larger than the maximum transverse dimensions by a factor of at least 10.

12. The method of claim 1 wherein the magnetic nanowires have maximum transverse dimensions of 20 to 500 nanometers and maximum longitudinal dimensions of 500 nanometers to 50 micrometers.

13. The method of claim 1 wherein the nanowires and the biological cells are attached by inclusion of the nanowires within the cells.

14. The method of claim 6 wherein the additional magnetic field is substantially perpendicular to the direction of fluid flow.

15. The method of claim 2 including the steps of trapping cells of one type and, after trapping the cells, the additional step of reversing the additional magnetic field to trap cells of a second type.

16. The method of claim 3 including the step of controlling the fluid flow rate to preferentially trap multiple cell clusters.

17. The method of claim 1 including the step of controlling the fluid flow rate to permit sedimentation of the nanowire carriers in head-to-tail N, S chains.

18. Apparatus for manipulating and trapping biological cells comprising:
a surface having disposed thereon a plurality of magnets arranged in a pattern to form a desired distribution of magnetic field strength over the surface including one or more regions of relatively high field strength;
a plurality of magnetic nanowires for attachment with the biological cells;
a fluid inlet to flow fluid comprising immersed magnetic nanowires over the surface; and
a fluid outlet to permit exit of the flowing fluid.

19. The apparatus of claim 18 wherein the magnets comprise microscale magnets having maximum dimensions of less than one millimeter in each of the three dimensions.

20. The apparatus of claim 18 wherein the magnets comprise an array of spaced apart magnets of opposite magnetic polarity.

21. The apparatus of claim 18 wherein the magnetic nanowires have a maximum transverse dimensions of less than one micron and maximum longitudinal dimensions larger than the maximum transverse directions by at least a factor of 10.

22. The apparatus of claim 18 further comprising a magnet for applying an additional magnetic field across the surface.

23. The apparatus of claim 18 wherein the magnetic nanowires have a maximum transverse dimensions of 20 to 500 nanometers and maximum longitudinal dimensions of 500 nanometers to 50 micrometers.
